(19) United States
(12) Patent Application Publication
(10) Pub. No.: US 2026/0204385 A1
(43) Pub. Date: Jul. 16, 2026

(54) PRESSURE APPLICATION FOR ASSISTING IN LUNGS VENTILATION OF A SUBJECT VENTILATED BY A MECHANICAL VENTILATION

(71) Applicant: SHEBA IMPACT LTD., Ramat-Gan (IL)

(72) Inventors: Itai PESSACH, Raanana (IL); Gil SOKOL, Ganei Tikva (IL); Moshe ASHKENAZI, Ganei Tikva (IL)

(21) Appl. No.: 19/133,419

(22) PCT Filed: Nov. 28, 2023
(86) PCT No.: PCT/IL2023/051221

§ 371 Date: May 28, 2025

(30) Foreign Application Priority Data
Nov. 28, 2022 (LU) LU503115

(51) Int. Cl. G16H 20/40; A61H 31/00; G16H 50/30

(52) U.S. Cl. CPC G16H 20/40; A61H 31/00; G16H 50/30; A61H 2201/107; A61H 2201/5007; A61H 2230/405

(57) ABSTRACT

The present disclosure provides a unique solution for assisting in the ventilation of lungs of subject that is performed by artificial mechanical ventilator. The solution of the present invention improves the efficiency of the ventilation effect by applying desired pressure profile on portions of the upper body part of the subject in synchronization with the mechanical ventilator operation, thereby creating a desired pressure profile or gradient in the lungs airways to ensure that air pumped into the lungs by the mechanical ventilator spreads more homogeneously in the lungs than without the intervention of the system of the present invention. The application of pressure is determined after analyzing data from sensors of the system that are placed on a plurality of portions on the upper body part of the subject and data received from the mechanical ventilator. These sensors sense data indicative of the ventilation profile within the lungs, i.e. pressure profile along different portions of the lung. The system provides extra-thoracic pressure on the rib cage according to pre-defined and adaptive algorithms based on the physiological status, the respiratory mechanics, and the inhomogeneous nature of lung injury. By applying pressure to the hyperventilated areas and reducing the transpulmonary pressure gradient, the system prevents hyperinflation trauma to the open areas and atelecto-trauma to the closed areas. The system is synchronized with the mechanical ventilator and adapt to changes in the patient's respiratory mechanics.

### TECHNOLOGICAL FIELD

The present disclosure is in the field of respiratory assisting techniques, in particular techniques in combination with mechanical ventilators.

### BACKGROUND ART

References considered to be relevant as background to the presently disclosed subject matter are listed below:
WO 2021/214769
US 2011/313332
WO 2019/240665
US 2021/008309
U.S. Pat. No. 10,945,699

Acknowledgement of the above references herein is not to be inferred as meaning that these are in any way relevant to the patentability of the presently disclosed subject matter.

### GENERAL DESCRIPTION

The present disclosure provides a unique solution for assisting in the ventilation of lungs of subject that is performed by artificial mechanical ventilator such as high-frequency jet ventilators, neonatal ventilators, positive airway pressure ventilators, continuous positive airway pressure ventilators, transport ventilators, intensive-care ventilators or the like. The solution of the present invention improves the efficiency of the ventilation effect by applying desired pressure profile on portions of the upper body part of the subject in synchronization with the mechanical ventilator operation, thereby creating a desired pressure profile or gradient in the lungs airways to ensure that air pumped into the lungs by the mechanical ventilator spreads more homogeneously in the lungs than without the intervention of the system of the present invention. The application of pressure is determined after analyzing data from sensors of the system that are placed on a plurality of portions on the upper body part of the subject. Various types of sensors may be used to sense relevant data, including, but not limited to, auditory, vibration, force, piezo and others. These sensors sense data indicative of the ventilation profile within the lungs, i.e. pressure profile along different portions of the lung. For example, if the sensors sense signals that indicates a pressure gradient above a desired threshold between two parts of the lungs, which can mean an obstruction in an airway within the lungs, an external pressure by pressure applicators of the system is applied to reduce the gradient to a desired level to allow efficient ventilation of the two portions.

The system provides extra-thoracic pressure on the rib cage according to pre-defined and adaptive algorithms based on the physiological status, the respiratory mechanics, and the inhomogeneous nature of lung injury. By applying pressure to the hyperventilated areas and reducing the transpulmonary pressure gradient, the system prevents hyperinflation trauma to the open areas and atelecto-trauma to the closed areas. The system is synchronized with the mechanical ventilator and adapt to changes in the patient's respiratory mechanics.

Therefore, an aspect of the present disclosure provides a system for assisting in mechanical ventilation of a subject. The system comprising an array of pressure applicators to be fitted over different body portions of an upper body part of the subject, i.e. the torso, the back, the side of the upper body and/or the abdomen so as to apply pressure on the rib cage. Namely, the reference to the upper body part of the subject should be interpreted as any part above the waist and below the head. The pressure applicators are configured to controllably apply pressure on said body portions, namely to apply a desired level of pressure within certain time frames in response to controlled commands from a processing circuitry or a controller.

The system further comprising at least one processing circuitry being configured and operable for: (i) receiving lungs ventilation data indicative of ventilation profile in the lungs of the subject, namely a mapping of areas in the lungs that are hyper ventilated, areas that are hypo ventilated and areas that are normally ventilated; (ii) receiving mechanical ventilator data from a mechanical ventilator ventilating the subject, the ventilator data is indicative of the ventilation profile provided by the mechanical ventilator to the subject and may include any parameter that is recorded by the mechanical ventilator during the ventilation of the subject; (iii) analyzing said lungs ventilation data and the mechanical ventilator data and generating a first pressure application profile based thereon. Namely the pressure application profile to be executed on the body of the subject depends on parameters received from the mechanical ventilator and parameters related to the mapping of the ventilation profile in the lungs of the subject. The combination of the data of the obstructed and non-obstructed areas of the lungs of the subject and the data of the mechanical ventilator' ventilation profile allows the processing circuitry to plan and generate the pressure application profile that may potentially yield the optimal result of the ventilation of the lungs of the subject by the mechanical ventilator; (iv) executing the pressure application profile by the array of pressure applicators to thereby apply said desired pressure application profile on the subject, namely transmitting signals to the pressure applicators of the array to result in their pressure application on different portions of the upper body part and the generation of the desired pressure profile application on the upper body part.

It is to be noted that any combination of the described embodiments with respect to any aspect of this present disclosure is applicable. In other words, any aspect of the present disclosure can be defined by any combination of the described embodiments.

In some embodiments, the system further comprises one or more memories coupled to the at least one processing circuitry and storing programming instructions for execution by the at least one processing circuitry the operations defined that it is configured to perform, e.g. the above operations (i)-(iv).

In some embodiments of the system, the processing circuitry is further configured to receive vital signs data of the subject comprises at least one of: heart rate, oxygen saturation and respiratory rate. The analysis and the generation of the first pressure application profile is also based on said vital signs data of the subject.

In some embodiments of the system, said lungs ventilation data comprises at least one of: obstruction sites in the lungs derived from sensing ventilation, rhonchus, thorax expansion profile, diaphragm movement profile, auscultation with microphone, breath sounds, rhonchi, or any combination thereof.

In some embodiments of the system, said mechanical ventilator data comprises at least one of: tidal volume, mean airway pressure, peak pressure, Positive end-expiratory pressure (PEEP), inspiratory airflow, intrinsic PEEP, plateau pressure, driving pressure, transpulmonary pressure, mechanical energy, and mechanical power and intensity, resistance, compliance and any other parameters that are measured by the mechanical ventilator, or any combination.

In some embodiments of the system, the first pressure application profile comprises temporal variation of pressure application intensity on different body portions in synchronization with the mechanical ventilator operation. This is important to ensure that while the air is introduced into the lungs by the mechanical ventilator, the desired pressure is applied on the upper body part of the subject. The first pressure application profile may be updated at each ventilation cycle or within a certain number of ventilation cycles that can be predetermined or can be continuously updated.

In some embodiments of the system, the first pressure application profile comprises temporal variation of pressure application intensity on different body portions in synchronization with the inspiratory phase of the mechanical ventilator. Namely, the pressure application performed by the array of pressure applicators is limited to be performed only during the inspiratory phase of the mechanical ventilator. Therefore, the processing circuitry is configured to execute the first pressure application profile based on data received from the mechanical ventilator such that the pressure application occurs simultaneously to the inflow of air by the mechanical ventilator to the lungs of the subject.

In some embodiments of the system, the first pressure application profile comprises temporal variation of pressure application intensity on different body portions in synchronization with the expiratory phase of the mechanical ventilator. Namely, the pressure application performed by the array of pressure applicators is limited to be performed only during the expiratory phase of the mechanical ventilator. Therefore, the processing circuitry is configured to execute the first pressure application profile based on data received from the mechanical ventilator such that the pressure application occurs simultaneously to the outflow of air from the lungs of the subject. By applying pressure during the expiratory phase of the mechanical ventilator, the areas in the lungs on which the pressure is applied on are blocked or at least partially blocked from receiving air from the mechanical ventilator so as to allow the air to better distribute in the lungs.

In some embodiments of the system, the processing circuitry is further configured to analyze said lungs ventilation data to identify hyperventilated areas in the lungs to be reduced with their received airflow during ventilation, namely during inflow of air by the mechanical ventilation, and execute said mechanical ventilation profile in synchronization with the expiratory phase of the mechanical ventilator to press on the upper body parts that are associated with said hyperventilated areas to reduce their received airflow in the following ventilation process performed by the mechanical ventilator. This causes some degree of blockage of these hyperventilated areas and ensures that the inflow air from the mechanical ventilator spreads more homogeneously in the lungs to obtain a more efficient ventilation process.

In some embodiments of the system, the first pressure application profile comprises applying pressure on different body portions during the phase of pushing air by the mechanical ventilator into the lungs. It is to be noted that the application of pressure can be completely overlapping with the positive pressure application of the mechanical ventilator causing inflow of air into the lungs or just a part of that phase.

In some embodiments of the system, said first pressure application profile is generated so as to result in maintaining the transpulmonary pressure in selected areas of the lungs within a desired range. Namely, a pressure map of the airways in the lungs of the subject may be generated and continuously updated by the sensed data of the sensors. For each area of the lungs there can be a specific desired transpulmonary pressure range and when the transpulmonary pressure of the specific area is sensed to be outside this range, one or more pressure applicators that are associated with this area are operated to apply a required pressure to obtain the desired transpulmonary pressure range in the specific area.

In some embodiments of the system, said first pressure application profile is generated so as to result in a pressure difference lower than a certain threshold between selected areas of the lungs. Namely, the first pressure application profile is determined in response to the sensed pressure within the lungs to reduce high pressure gradients between different areas of the lungs to obtain more homogeneous ventilation.

In some embodiments of the system, said first pressure application profile comprises at least one of: chest and/or abdominal pressure application state (simulating lying on the belly), back pressure application state (simulating lying on the back), side pressure application state (simulating lying on one side), or any combination thereof. Therefore, the first pressure application profile may simulate a rest body position of the subject, even if the subject is not in said rest body position. For example, the subject may lie on the back and the first pressure application profile may simulate that the subject is lying on his/her abdomen. This eliminates the need of turning the subject between different positions to affect the ventilation profile of air ventilated into his/her lungs by the mechanical ventilator.

In some embodiments of the system, the array of pressure applicators comprises a combination of soft and firm pressure applicators.

In some embodiments of the system, the processing circuitry is in data communication with a mechanical ventilator ventilating the subject and is configured to execute a mechanical ventilation profile of the subject by the mechanical ventilator in synchronization with the first pressure application profile to obtain a desired lungs ventilation profile of the subject.

In some embodiments of the system, said mechanical ventilation profile is determined based on the lungs ventilation data, namely forming a closed loop of control with the mechanical ventilation.

In some embodiments of the system, said desired lungs ventilation profile is determined to remove secretions from the air pathways in the lungs.

In some embodiments of the system, the first pressure application profile is periodical, the time period duration of a cycle is correlative to a time period of a cycle of ventilation performed by the mechanical ventilator.

In some embodiments of the system, said first pressure application profile comprises periodical cycles, each cycle comprises pressure application period, in which application of pressure is applied by the pressure applicators on the subject, and neutral period, in which no application of pressure is applied by the pressure applicators on the subject. The pressure application period is about the same duration as an inflow ventilation period of the mechanical ventilator, therefore, the application of the pressure by the pressure applicators is performed during an inflow of air into the lungs of the subject.

The term “about” should be interpreted as a deviation of ±20% of the nominal value. For example, if the value is about 10, thus it should be understood to be in the range of 8-12.

In this specific example, the term “about” should be understood as a range between ±20% of the inflow ventilator period (the period in which air is introduced into the lungs by the mechanical ventilator).

In some embodiments of the system, the first pressure application profile comprises application of pressure on said body portions of between about 30 cmH₂O and 60 cmH₂O, or more preferably, between about 40 cmH₂O and 50 cmH₂O.

In some embodiments, the system comprising additional sensors to sense thorax expansion profile and diaphragm movement profile.

In some embodiments, the system, further comprises a sensor array that comprises a plurality of sensing elements configured for sensing lungs ventilation activity of the subject and generating said lungs ventilation data indicative of ventilation profile in the lungs of the subject, namely to sense areas in the lungs that are experiencing hyper ventilation and other areas that may be experiencing hypo ventilation. The sensing elements may be placed in proximity to the pressure applicators or in different locations over the upper body part. For example, the system may have an array of modules, and each module may comprise at least one sensing element and at least one pressure applicator.

In some embodiments of the system, the sensor array comprises at least one of: pressure sensor, a microphone, a volumeter, an impedance sensor, an imaging system, a strain gauge, sensors for lung auscultation or any combination thereof.

In some embodiments of the system, said array of pressure applicators comprises at least one of: an inflatable sac, a fillable pad, an electrically activated pad, a manually adjustable belt, an automatically adjustable belt, a stretchable strap, or any combination thereof.

In some embodiments of the system, the processing circuitry is further configured to analyzing said lungs ventilation data, following an execution of said first pressure application profile and executing a third pressure application profile based on said lungs ventilation data. The analysis of the lungs ventilation data can be made on data in a selected time window following the execution of said first pressure application profile. The time window can be any selected time window, e.g. a time window that starts several minutes after the initial execution of the said first pressure application profile and may last for several minutes. If a ventilation of an obstructed lungs area is identified, the processing circuitry is configured to performing a third pressure adjustment in said first pressure application profile to obtain the third pressure application profile, and if no change of ventilation of obstructed lungs areas is identified, the processing circuitry is configured to performing a fourth pressure adjustment in said first pressure application profile to obtain the third pressure application profile.

In some embodiments of the system, the processing circuitry is further configured to profiling the mechanical ventilator data to obtain a mechanical ventilator profile, following an execution of said first pressure application profile and executing a second pressure application profile based on said mechanical ventilator profile. The analysis of the mechanical ventilator data can be made on data in a selected time window following the execution of said first pressure application profile. The time window can be any selected time window, e.g. a time window that starts several minutes after the initial execution of the said first pressure application profile and may last for several minutes. If an improvement variation is identified in the mechanical ventilator profile, the processing circuitry is configured to performing a first pressure adjustment in said first pressure application profile to obtain the second pressure application profile, and if a non-improvement variation is identified in the mechanical ventilator profile, the processing circuitry is configured to performing a second pressure adjustment in said first pressure application profile to obtain the second pressure application profile. The mechanical ventilator profile can be based on a time window that starts short after the execution of the first pressure application profile or several minutes after the execution of the first pressure application profile and lasts for several minutes, e.g. 1-10 minutes or 3-5 minutes.

In some embodiments of the system, said first pressure adjustment and third pressure adjustment comprise an increase of pressure intensity application on one or more body portions with respect to the first pressure application profile. This increase can be at any time point at the temporal pressure application profile of the first pressure application profile. In some embodiments, the first pressure adjustment and the third pressure adjustment are the same.

In some embodiments of the system, said second pressure adjustment and fourth pressure adjustment comprise a decrease of pressure intensity application on one or more body portions with respect to the first pressure application profile. Namely, the second pressure application profile comprises application of an increased pressure on at least one body portion with respect to the first pressure application profile. this can be at any time point at the temporal pressure application profile of the first pressure application profile. In some embodiments, the second pressure adjustment and the fourth pressure adjustment are the same.

It is to be noted that the terms “first”, “second”. “third” and “fourth” with respect to either the term “pressure application profile” or the term “pressure adjustment” should be understood merely as distinguishing terms and they do not necessarily indicate a certain order.

In some embodiments of the system, said profiling comprises profiling at least one mechanical ventilator parameter selected from: tidal volume, minute volume, ventilation volume, peak inspiratory pressure (PIP), PEPP, plateau pressure (Pplat), fraction of inspired oxygen (FiO₂) compliance, respiratory airway resistance (Res), PEEP, oxygen saturation (O₂Sat). Said improvement variation or said non-improvement variation are identified for at least one mechanical ventilator parameter. For example, improvement variation can be if higher compliance, lower Res, higher tidal volume higher O₂Sat, or ventilation of obstructed areas is identified.

Yet another aspect of the present disclosure provides a method for assisting in mechanical ventilation of a subject. The method comprising: (i) receiving lungs ventilation data indicative of ventilation profile in the lungs of the subject, namely a mapping of areas in the lungs that are hyper ventilated, areas that are hypo ventilated and areas that are normally ventilated; (ii) receiving mechanical ventilator data from a mechanical ventilator ventilating the subject, said ventilator data is indicative of the ventilation profile provided by the mechanical ventilator to the subject; (iii) analyzing said lungs ventilation data and the mechanical ventilator data and generating a first pressure application profile based thereon to be applied on different portions of an upper body part of the subject; and (iv) executing the first pressure application profile by an array of pressure applicators to thereby apply said first pressure application profile on the subject. It is to be noted that the order of the steps of the method can be interchanged and is not necessarily restricted to the order they described above.

In some embodiments, the method further comprising receiving vital signs data of the subject comprises at least one of: heart rate, oxygen saturation and respiratory rate. Said analyzing and generating is also based on said vital signs data of the subject.

In some embodiments of the method, said lungs ventilation data comprises at least one of: obstruction sites in the lungs derived from sensing rhonchus, thorax expansion profile, diaphragm movement profile, auscultation with microphone, breath sounds, rhonchi, or any combination thereof.

In some embodiments of the method, said mechanical ventilator data comprises at least one of: tidal volume, mean airway pressure, peak pressure, Positive end-expiratory pressure (PEEP), inspiratory airflow, intrinsic PEEP, plateau pressure, driving pressure transpulmonary pressure, mechanical energy, and mechanical power and intensity, resistance, compliance, and any other parameters that are measured by the mechanical ventilator or any combination thereof.

In some embodiments of the method, the first pressure application profile comprises applying pressure on different body portions in synchronization with the mechanical ventilator operation.

In some embodiments of the method, the first pressure application profile comprises temporal variation of pressure application intensity on different body portions in synchronization with the inspiratory phase of the mechanical ventilator. Namely, the pressure application performed by the array of pressure applicators is limited to be performed only during the inspiratory phase of the mechanical ventilator and occurs simultaneously to the inflow of air by the mechanical ventilator to the lungs of the subject.

In some embodiments of the method, the first pressure application profile comprises temporal variation of pressure application intensity on different body portions in synchronization with the expiratory phase of the mechanical ventilator. Namely, the pressure application performed by the array of pressure applicators is limited to be performed only during the expiratory phase of the mechanical ventilator and occurs simultaneously to the outflow of air from the lungs of the subject. By applying pressure during the expiratory phase of the mechanical ventilator, the areas in the lungs on which the pressure is applied on are blocked or at least partially blocked from receiving air from the mechanical ventilator so as to allow the air to better distribute in the lungs.

In some embodiments, the method further comprising analyzing said lungs ventilation data to identify hyperventilated areas in the lungs to be reduced with their received airflow during ventilation and execute said mechanical ventilation profile in synchronization with the expiratory phase of the mechanical ventilator to press on the upper body parts associated with said hyperventilated areas to reduce their received airflow in the following ventilation process performed by the mechanical ventilator. This causes some degree of blockage of these hyperventilated areas and ensures that the inflow air from the mechanical ventilator spreads more homogeneously in the lungs to obtain a more efficient ventilation process.

In some embodiments of the method, the first pressure application profile comprises applying pressure on different body portions during the phase of pushing air by the mechanical ventilator into the lungs.

In some embodiments of the method, said first pressure application profile is generated so as to result in maintaining a transpulmonary pressure in selected areas of the lungs within a selected range and/or to result in a pressure difference lower than a certain threshold between selected areas of the lungs.

In some embodiments of the method, said first pressure application profile comprises at least one of: chest and/or abdominal pressure application state (simulating lying on the belly), back pressure application state (simulating lying on the back), side pressure application state (simulating lying on one side), or any combination thereof. Therefore, the first pressure application profile may simulate a rest body position of the subject, even if the subject is not in said rest body position. For example, the subject may lie on the back and the first pressure application profile may simulate that the subject is lying on his/her abdomen.

In some embodiments, the method further comprising executing a mechanical ventilation profile of the subject by the mechanical ventilator ventilating the subject in synchronization with the first pressure application profile to obtain a desired lungs ventilation profile of the subject.

In some embodiments of the method, said mechanical ventilation profile is determined based on the lungs ventilation data, namely forming a closed loop of control with the mechanical ventilation.

In some embodiments of the method, the first pressure application profile is periodical, the time period duration of a cycle is correlative to a time period of a cycle of ventilation performed by the mechanical ventilator.

In some embodiments of the method, said first pressure application profile comprises periodical cycles, each cycle comprises pressure application period, in which application of pressure is applied by the pressure applicators on the subject, and neutral period, in which no application of pressure is applied by the pressure applicators on the subject. The pressure application period is about the same duration as an inflow ventilation period of the mechanical ventilator.

In some embodiments of the method, the first pressure application profile comprises application of pressure on said body portions of between about 30 cmH₂O and 60 cmH₂O, or more preferably, between about 40 cmH₂O and 50 cmH₂O.

In some embodiments, the method further comprises sensing lungs ventilation activity of the subject and generating said lungs ventilation data indicative of ventilation profile in the lungs of the subject.

In some embodiments of the method, said sensing comprises sensing with a sensor array.

In some embodiments of the method, the sensor array comprises at least one of: pressure sensor, a microphone, a volumeter, an impedance sensor, an imaging system, a strain gauge, sensors for lung auscultation or any combination thereof.

In some embodiments of the method, the array of pressure applicators comprises at least one of: an inflatable sac, a fillable pad, an electrically activated pad, a manually adjustable belt, an automatically adjustable belt, a stretchable strap, or any combination thereof.

In some embodiments, the method further comprises profiling the mechanical ventilator data to obtain a mechanical ventilator profile, following an execution of said first pressure application profile and executing a second pressure application profile based on said mechanical ventilator profile. The analysis of the mechanical ventilator data can be made on data in a selected time window following the execution of said first pressure application profile. The time window can be any selected time window, e.g. a time window that starts several minutes after the initial execution of the said first pressure application profile and may last for several minutes. If an improvement variation is identified in the mechanical ventilator profile, the method further comprises performing a first pressure adjustment in said first pressure application profile to obtain the second pressure application profile, and if a non-improvement variation is identified in the mechanical ventilator profile, the method further comprises performing a second pressure adjustment in said first pressure application profile to obtain the second pressure application profile. The mechanical ventilator profile can be based on a time window that starts right after the execution of the first pressure application profile or several minutes after the execution of the first pressure application profile and lasts for several minutes, e.g. 1-10 minutes or 3-5 minutes.

In some embodiments, the method further comprises analyzing said lungs ventilation data, following said executing of said first pressure application profile and executing a third pressure application profile based on said lungs ventilation data. The analysis of the lungs ventilation data can be made on data in a selected time window following the execution of said first pressure application profile. The time window can be any selected time window, e.g. a time window that starts several minutes after the initial execution of the said first pressure application profile and may last for several minutes. If a ventilation of an obstructed lungs area is identified, method further comprises performing a third pressure adjustment in said first pressure application profile to obtain the third pressure application profile, and if no change of ventilation of obstructed lungs areas is identified, the method further comprises performing a fourth pressure adjustment in said first pressure application profile to obtain the third pressure application profile.

In some embodiments of the method, said first and third pressure adjustments comprise an increase of pressure intensity application on one or more body portions with respect to the first pressure application profile. This can be at any time point at the temporal pressure application profile of the first pressure application profile. In some embodiments, the first and third pressure adjustments are the same.

In some embodiments of the method, said second and fourth pressure adjustment comprise a decrease of pressure intensity application on one or more body portions with respect to the first pressure application profile. Namely, the second pressure application profile comprises application of an increased pressure on at least one body portion with respect to the first pressure application profile. this can be at any time point at the temporal pressure application profile of the first pressure application profile. In some embodiments, the second and fourth pressure adjustments are the same.

It is to be noted that the terms “first”, “second”. “third” and “fourth” with respect to either the term “pressure application profile” or the term “pressure adjustment” should be understood merely as distinguishing terms and they do not necessarily indicate a certain order.

In some embodiments of the method, said profiling comprises profiling at least one mechanical ventilator parameter selected from: tidal volume, minute volume, ventilation volume, peak inspiratory pressure (PIP), PEPP, plateau pressure (Pplat), fraction of inspired oxygen (FiO₂) compliance, respiratory airway resistance (Res), PEEP, oxygen saturation (O₂Sat); wherein said improvement variation or said non-improvement variation are identified for at least one mechanical ventilator parameter. For example, improvement variation can be if higher compliance, lower Res, higher tidal volume higher O₂Sat, or ventilation of obstructed areas is identified.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a block diagram of exemplifying a non-limiting example of an embodiment of the system according to an aspect of the present disclosure.

FIG. 2 is a schematic illustration of a non-limiting example of the modules of the system placed over an upper body portion of a subject according to an aspect of the present disclosure.

FIG. 3 is a flow diagram exemplifying an operation cycle employing the invention of the present disclosure.

### DETAILED DESCRIPTION

Mechanical ventilation is a mean to supply oxygenated air in positive pressure. This is a common therapy to support patients with respiratory failure and in other life-threatening situations, or to assist patients with respiratory illnesses.

However, the implementation of mechanical ventilation significantly changes the physiology of respiration and results in changes to the lungs and respiratory system that may, sometimes, result in extensive damage and severe complications. It has been shown that the extent of the pulmonary damage resulting from mechanical ventilation is linked to the levels of the pressure used, the duration of ventilation, comorbidities of the patient as well as many other factors.

The distribution of pressures and volumes supplied during mechanical ventilation in different areas of the lungs is inhomogeneous due to differences in airway resistance and tissue compliance in various areas of the lung. Therefore, some areas of the lung may become hyperventilated and other areas hypoventilated. Furthermore, depending on the patients'posture, the ability to clear secretions, and the variable compliance of different lung areas may result in local atelectasis (lung collapse). These changes play a key role in the mechanisms leading to barotrauma, volutrauma, atelectotrauma and inflammation that lead to significant pulmonary damage.

A key mechanism in this process is the transpulmonary pressure which reflects the pressure in the airways (ventilation pressure) minus the pressure outside of the thorax (atmospheric pressure). When elevated this pressure is the major culprit leading to pulmonary damage.

Controlling the transpulmonary pressure in various areas of the lung is a key strategy in lung—protective ventilation.

The present invention differentially reduces the transpulmonary pressure in designated areas of the lung by focally elevating the pressure outside of the thorax. This is achieved by applying external pressure to the thoracic wall (front and back) and abdomen. The device is synchronized with the ventilator and responds to changes in the patient respiratory mechanics. The device may include several pressure modules (for example, 5-9 to each lung as well as abdominal modules) that can provide external pressure to the thoracic wall and abdomen in a sufficient resolution. The present invention further includes sensors such as pressure, movement and mechanics monitors, microphones and other sensors, all may be part of the pressure modules or separated therefrom, that are capable of sensing and/or analyzing the status of the lung areas/airways and to identify whether these areas are hypo/hyper ventilated. The pressure application profile may be improved and personalized over time by AI algorithms.

The following figures are provided to exemplify embodiments and realization of the invention of the present disclosure.

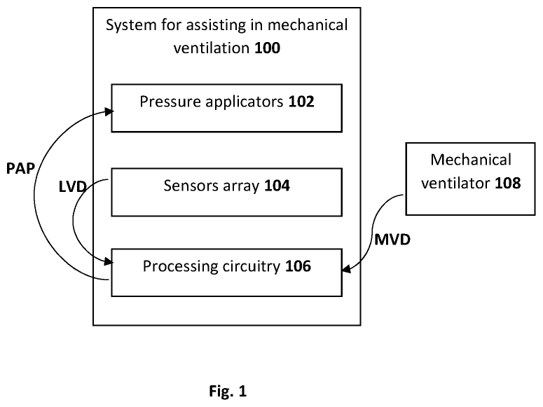

Reference is being made to FIG. 1, which is a block diagram of the system for assisting in mechanical ventilation of a subject according to an aspect of the present disclosure. The system 100 comprises an array of pressure applicators 102 configured to be placed on different locations of the upper body, such as the torso, the back, the side of the upper body and/or the abdomen so as to apply pressure on the rib cage. Each pressure applicator is associated with the location it is placed on, namely in case a pressure application on a specific location is required, the specific pressure applicator is activated to apply pressure on said specific desired location.

The system further comprises a sensors array 104, each sensor of the array is configured to be placed on a different location of the upper body of the subject and to sense a parameter indicative of the lungs ventilation profile associated with the external location where the sensor is positioned. With the sensing data obtained by all the sensors, a map of lungs ventilation profile can be generated and lungs ventilation data LVD indicative of the lungs ventilation profile is transmitted to a processing circuitry 106 of the system 100 to analyze the data and determine the desired pressure profile to be executed by the pressure applicators 102.

The processing circuitry 106 is configured to receive mechanical ventilation data MVD from a mechanical ventilator 108 that ventilates the subject. The mechanical ventilation data may include any of the following parameters: tidal volume, mean airway pressure, peak pressure, Positive end-expiratory pressure (PEEP), inspiratory airflow, intrinsic PEEP, plateau pressure, driving pressure, transpulmonary pressure, mechanical energy, and mechanical power and intensity, resistance, compliance, any other data piece that is measure by the mechanical ventilator, or any combination thereof. The processing circuitry 106 is configured to process the mechanical ventilation data MVD obtained from the mechanical ventilator 108 and the lungs ventilation data LVD obtained from the sensors array 104 and determine based thereon the pressure application profile PAP required to be executed by the pressure applicators 102 in order to optimize the ventilation of the mechanical ventilator 108 and reduce the risk of damage to the lungs. The pressure application profile PAP is determined so as to be in synchronization with the operation of the mechanical ventilator 108 such that hyperventilated areas in the lungs will receive less ventilation and hypoventilated areas in the lungs will receive more ventilation, thereby electively reducing transpulmonary pressure gradients. For example, the pressure application profile PAP may be periodical with a period time matching to the period time of the ventilation provided by the mechanical ventilator or the respiration rate of the subject. While the mechanical ventilator introduce air into the lungs, a pressure is applied according to the identification of blockage of airways and the pressure may be relived while the phase of introduction of air into the lungs is over.

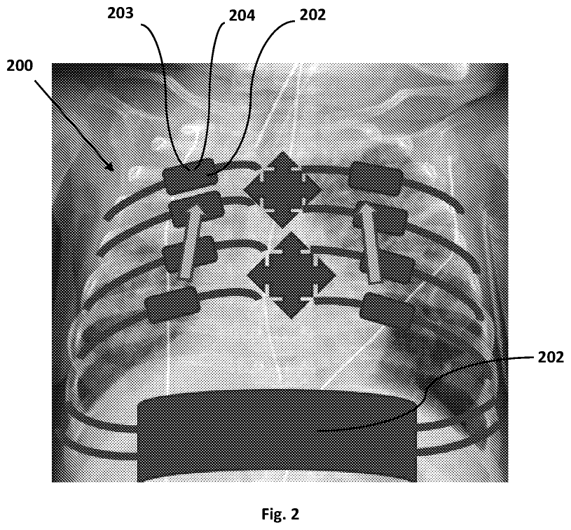

In the figures throughout the application, like elements of different figures were given similar reference numerals shifted by the number of hundreds corresponding to the number of the respective figure. For example, element 202 in FIG. 2 serves the same function as element 102 in FIG. 1.

FIG. 2 is a schematic illustration of a non-limiting embodiment exemplifying the wearable components of the system placed around the upper body portion of the subject. In this example, the system 200 comprises a plurality of modules 203, each module 203 is configured to be placed on a different part of the front or back of the upper body portion of the subject. Each module 203 comprises at least one pressure applicator 202 and at least one sensor 204 for sensing lungs ventilation data indicative of the transpulmonary pressure map of the subject. The pressure applicators 202 are configured to execute a desired pressure application profile, namely a pressure application scheme of pressure intensity over time in a plurality of location of the upper body of the subject, to reduce inhomogeneity of air ventilation by a mechanical ventilator to a subject. The pressure applicators are configured to apply pressure on the locations of the thoracic wall that are associated with parts in the lungs in which the pressure is relatively high and is required to be reduced to avoid hypoventilation.

The processing circuitry (not shown) is configured to receive the lungs ventilation data from the sensors 204 and the mechanical ventilation data from the mechanical ventilator (not shown) to generate the temporal pressure application profile that includes pressure application variability over time, to reduce pressure gradient within the lungs and increase the ventilation homogeneity of the ventilation of the mechanical ventilator according to a desired ventilation scheme.

The system provides a complementary system to the mechanical ventilator that is synchronized with it. While common approach is to minimize the positive pressure of the ventilation, the system of the present invention provides a different approach that includes increasing the extra-thoracic pressure. By providing external pressure to the thorax and abdomen the trans-pulmonary pressure gradient is selectively reduced over various areas of the lung and hence better balance the air distribution in the lung, minimize V/Q (the V/Q ratio is the amount of air that reaches your lungs divided by the amount of blood flow in the capillaries in your lungs) mismatch and reduce lung injury.

Furthermore, the system of the present invention is unique by providing an adaptive solution that selectively addresses the variable mechanics of different lung areas, as well as continuously responds to changes in respiratory mechanics in synchrony with the mechanical ventilator.

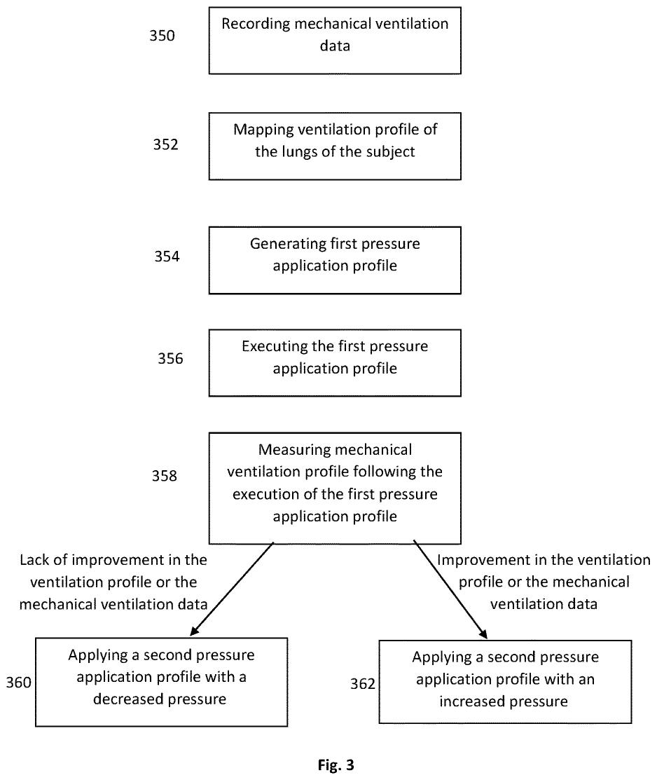

Reference is now being made to FIG. 3, which is a flow diagram exemplifying an operation cycle employing the invention of the present disclosure. The cycle starts with recording mechanical ventilation data 350 and mapping ventilation profile of the lungs of the subject 352. Parameters that may be recorded as part of the mechanical ventilation data are at least tidal volume (Vt), Minute Volume, PIP, PEPP, Pplat, FiO₂ Comp, Res, PEEP, O₂Sat, Ventilation mode. The ventilation profile of the lungs of the subject are mapped by at least one of the following: auscultation, imaging (e.g. CT/US/Rx), microphone, Vibration Sensors, or any other sensors (US based, movement, pressure, etc.). Therefore, the ventilation profile of the lungs of the subject, e.g. the acoustic data indicative of the air ventilation of the lungs of the subject can be collected, for example, manually via statoscope or automatically via an array of microphones. This allows the identification of areas of normal ventilation, reduced ventilation or over ventilation. Optionally, the decision on what chest area to apply the pressure is guided based on a CT scan (or closed loop based on any other imagining data) that is taken on daily basis, and illustrates visually the open/closed lung areas. The parameters of the mechanical ventilation data and the mapping ventilation profile indicate together the requirements for the pressure profile to be applied on the body of the subject in order to improve the ventilation of his/her lungs. Therefore, the cycle continues with generating first application profile 354 to be applied on the body of the subject. The first application profile is typically applied in synchronization with the mechanical ventilator that ventilates the subject, namely the first pressure application profile is a temporal profile that may include a period of increase of the pressure, a period of steady application of pressure and a period of decrease of the pressure applied on the subject. A typical pressure range that is applied on the subject is between 5-50 cmH₂0. After generating the first pressure application profile, the cycle proceeds with executing the first pressure application profile 356. The first pressure application profile typically comprises cyclic and repetitive application of variable external thoracic pressure starting at 10 cmH₂O over specific lung segments for up to 10 minutes. Following the execution of the first pressure application profile, either right after the execution or after a several execution cycles, the operation cycle proceeds with measuring mechanical ventilation profile 358 to identify whether at least one parameter of the ventilation of the lungs of the subject improved. Typically, this is performed by measuring and analyzing the parameters of the mechanical ventilator, but this can also include mapping the ventilation profile of the lungs of the subject and identifying whether areas that were obstructed are now ventilated. If lack of improvement is identified, the operation cycle further comprises applying a second pressure application profile with a decreased pressure 360. If an improvement is identified, the operation cycle further comprises applying a second pressure application profile with an increased pressure 362. An example of identification of improvement is if at least one of higher compliance, lower Res, higher Vt, higher O₂Sat, or ventilation of obstructed areas is identified. Each pressure application profile may be applied for a certain time frame, which can be between 1-10 minutes, 2-9 minutes, 3-8 minutes, 4-7 minutes, or 3-5 minutes. Typically, the maximum threshold pressure to be applied is about 100 cmH₂O.

1-51. (canceled)

52. A system for assisting in mechanical ventilation of a subject, comprising:
an array of pressure applicators for fitting over different body portions of an upper body part of the subject, said pressure applicators are configured to controllably apply pressure on said body portions;
at least one processing circuitry being configured and operable for:
(i) receiving said lungs ventilation data indicative of ventilation profile in the lungs of the subject;
(ii) receiving mechanical ventilator data from a mechanical ventilator ventilating the subject, said ventilator data is indicative of the ventilation profile provided by the mechanical ventilator to the subject;
(iii) analyzing said lungs ventilation data and the mechanical ventilator data and generating a first pressure application profile based thereon;
(iv) executing the pressure application profile by the array of pressure applicators to thereby apply said first pressure application profile on the subject.

53. The system of claim 52, wherein the processing circuitry is further configured to receive vital signs data of the subject comprises at least one of: heart rate, oxygen saturation and respiratory rate, wherein said analyzing is also based on said vital signs data of the subject.

54. The system of claim 52, wherein said lungs ventilation data comprises at least one of: obstruction sites in the lungs, auscultation with microphone, breath sounds, thorax expansion profile, diaphragm movement profile or any combination thereof;
wherein said mechanical ventilator data comprises at least one of: tidal volume, mean airway pressure, peak pressure, Positive end-expiratory pressure (PEEP), inspiratory airflow, intrinsic PEEP, plateau pressure, driving pressure, transpulmonary pressure, mechanical energy, and mechanical power and intensity, resistance, compliance or any combination thereof.

55. The system of claim 52, wherein the first pressure application profile comprises temporal variation of pressure intensity application on different body portions in synchronization with the mechanical ventilator operation;
wherein the first pressure application profile comprises temporal first pressure application intensity profile on different body portions during the phase of pushing air by the mechanical ventilator into the lungs.

56. The system of claim 52, wherein said first pressure application profile is generated so as to result in maintaining a transpulmonary pressure in selected areas of the lungs within a selected range.

57. The system of claim 52, wherein said first pressure application profile is generated so as to result in a pressure difference lower than a certain threshold between selected areas of the lungs.

58. The system of claim 52, wherein said first pressure application profile comprises at least one of: chest and/or abdominal pressure application state, back pressure application state, side pressure application state, or any combination thereof;
wherein said array of pressure applicators comprises a combination of soft and firm pressure applicators.

59. The system of claim 52, wherein the processing circuitry is in data communication with a mechanical ventilator ventilating the subject and is configured to execute a mechanical ventilation profile of the subject by the mechanical ventilator in synchronization with the first pressure application profile to obtain a desired lungs ventilation profile of the subject.

60. The system of claim 59, wherein said mechanical ventilation profile is determined based on the lungs ventilation data;
wherein said desired lungs ventilation profile is determined to remove secretions from the air pathways in the lungs.

61. The system of claim 59, wherein the processing circuitry is configured to execute said mechanical ventilation profile in synchronization with an expiratory phase of the mechanical ventilator.

62. The system of claim 61, wherein the processing circuitry is further configured to analyze said lungs ventilation data to identify hyperventilated areas in the lungs to be reduced with their received airflow during ventilation and execute said mechanical ventilation profile in synchronization with the expiratory phase of the mechanical ventilator to press on the upper body parts associated with said hyperventilated areas to reduce their received airflow in the following ventilation process.

63. The system of claim 52, wherein the first pressure application profile is periodical, the time period duration of a cycle is correlative to a time period of a cycle of ventilation performed by the mechanical ventilator.

64. The system of claim 52, wherein said first pressure application profile comprises periodical cycles, each cycle comprises pressure application period and neutral period, wherein the pressure application period is about the same duration as an inflow ventilation period of the mechanical ventilator.

65. The system of claim 52, comprising additional sensors to sense thorax expansion profile and diaphragm movement profile;
wherein the system further comprises a sensor array configured for sensing lungs ventilation activity of the subject and generating said lungs ventilation data indicative of ventilation profile in the lungs of the subject;
wherein the sensor array comprises at least one of: pressure sensor, a microphone, a volumeter, an impedance sensor, an imaging system, a strain gauge, sensors for lung auscultation or any combination thereof;
wherein said array of pressure applicators comprises at least one of: an inflatable sac, a fillable pad, an electrically activated pad, a manually adjustable belt, an automatically adjustable belt, a stretchable strap, or any combination thereof.

66. The system of claim 52, wherein the processing circuitry is further configured to analyzing said lungs ventilation data, following an execution of said first pressure application profile and executing a third pressure application profile based on said lungs ventilation data; wherein if a ventilation of an obstructed lungs area is identified, the processing circuitry is configured to performing a first pressure adjustment in said first pressure application profile to obtain the third pressure application profile, and if no change of ventilation of obstructed lungs areas is identified, the processing circuitry is configured to performing a second pressure adjustment in said first pressure application profile to obtain the third pressure application profile.

67. The system of claim 52, wherein the processing circuitry is further configured to profiling the mechanical ventilator data to obtain a mechanical ventilator profile, following an execution of said first pressure application profile and executing a second pressure application profile based on said mechanical ventilator profile; wherein if an improvement variation is identified in the mechanical ventilator profile, the processing circuitry is configured to performing a first pressure adjustment in said first pressure application profile to obtain the second pressure application profile, and if a non-improvement variation is identified in the mechanical ventilator profile, the processing circuitry is configured to performing a second pressure adjustment in said first pressure application profile to obtain the second pressure application profile.

68. The system of claim 67, wherein said first pressure adjustment comprises an increase of pressure intensity application on one or more body portions with respect to the first pressure application profile.

69. The system of claim 67, wherein said second pressure adjustment comprises a decrease of pressure intensity application on one or more body portions with respect to the first pressure application profile.

70. The system of claim 67, wherein said profiling comprises profiling at least one mechanical ventilator parameter selected from: tidal volume, minute volume, ventilation volume, peak inspiratory pressure (PIP), PEPP, plateau pressure (Pplat), fraction of inspired oxygen (FiO₂) compliance, respiratory airway resistance (Res), PEEP, oxygen saturation (O₂Sat); wherein said improvement variation or said non-improvement variation are identified for at least one mechanical ventilator parameter.

71. A method for assisting in mechanical ventilation of a subject, comprising:
receiving lungs ventilation data indicative of ventilation profile in the lungs of the subject;
receiving mechanical ventilator data from a mechanical ventilator ventilating the subject, said ventilator data is indicative of the ventilation profile provided by the mechanical ventilator to the subject;
analyzing said lungs ventilation data and the mechanical ventilator data and generating a first pressure application profile based thereon to be applied on different portions of an upper body part of the subject;
executing the pressure application profile an array of pressure applicators to thereby apply said desired pressure application profile on the subject.
